(19) United States
(12) Patent Application Publication
(10) Pub. No.: US 2022/0258279 A1
(43) Pub. Date: Aug. 18, 2022

(54) DEVICE AND METHOD FOR PROCESSING MATERIAL BY MEANS OF LASER RADIATION

(71) Applicant: NOVANTA EUROPE GMBH, Garching b. München (DE)

(72) Inventors: Markus Guggenmos, Schwandorf (DE); Martin Hartmann, Burgthann (DE); Thomas Buckert, Burgthann (DE); Fabian Mütel, Regensburg (DE)

(21) Appl. No.: 17/628,551

(22) PCT Filed: Jul. 20, 2020
(86) PCT No.: PCT/EP2020/070453

§ 371 Date: Jan. 19, 2022

(30) Foreign Application Priority Data
Jul. 24, 2019 (DE) 10 2019 120 010.5

(51) Int. Cl. B23K 26/0622; B23K 26/082

(52) U.S. Cl. CPC B23K 26/0624; B23K 26/0648; B23K 26/082

(57) ABSTRACT

The invention relates to a method for processing material, in particular for modifying material and/or material properties, by means of laser radiation, comprising the following steps: a) generating a multiplicity of laser pulses (L); b) controlling the point of impact of the laser pulses (L) on a workpiece (100) to be processed, in particular deflecting the laser pulses (L) and/or moving the workpiece (100) to be processed, such that the laser pulses (L) are guided along a predetermined trajectory (Z) on the workpiece (100) to be processed. According to the invention, —a pulse-to-pulse time interval (Δt) between the individual laser pulses (L) generated and/or—a pulse energy (Pi) of the laser pulses (L) and/or—a beam diameter (D) of the laser pulses (D) and/or—the predetermined trajectory (Z) is/are specifically subjected to noise.

The invention relates to a method for processing material, in particular for modifying material and/or material properties, by means of laser radiation, according to claim 1, as well as to a device for processing material, in particular for modifying material and/or material properties, by means of laser radiation, according to claim 7.

In processing material by laser, pulsed laser beams are sometimes guided and/or directed over materials to be processed such that the pulsed laser beams process the materials, for example, in particular modify material properties and/or remove material on this occasion.

In the state of the art, methods and devices for processing material by laser have been hitherto described, in which a particularly high degree of precision of the temporal courses of the individual laser pulses and/or the laser beam guidance and/or the performance level of the individual laser pulses is intended to be achieved, as described, for example, in the printed publications DE 102 45 717 A1, DE 10 2009 042 003 B4, and DE 11 2005 002 987 T5.

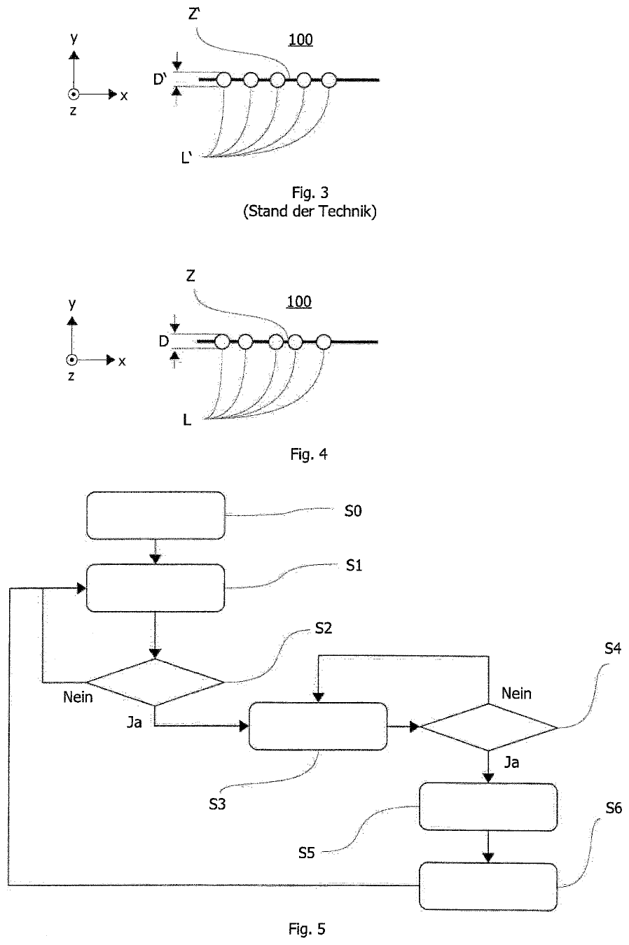

In FIG. 3, a top view of a workpiece 100 is shown during processing the workpiece according to a method of the state of the art. In the illustrated example, a pulsed laser beam is guided along a trajectory Z′ on a surface of the workpiece 100. In the depicted example, the trajectory Z′ runs along an x-axis of a coordinate system x, y, z, wherein the x-axis and y-axis span a workpiece plane of the workpiece 100, and the z-axis runs orthogonally to the x-axis and y-axis. Depending on the focus and quality of the material of the workpiece, it is also possible for the pulsed laser beam to be guided along a trajectory Z′ within the workpiece.

The processing points L′ produced by the laser pulses are moreover shown in FIG. 3, which have a diameter D′, wherein by continuously guiding and/or deflecting the laser beam in combination with a precise time interval of the individual laser pulses, the processing points L′ produced by the laser pulses are regularly spaced from one another along trajectory Z′. Consequently, a regular pattern of processing points L′ is generated.

In particular in materials employed in optics, through which light falls and/or is guided, the highly precise processing of the material, however, results in periodic structures being generated in the material and/or periodic structures of the material properties of the material being generated. Under the incidence of light, this results in undesired interference patterns and/or diffraction effects to be possibly generated.

The invention is based on the task of providing a method for processing material by laser, which is cost-efficient, on the one hand, and optimizes the optical properties of the workpiece to be processed, on the other hand. In particular, a method and a device for avoiding the previously described problems are intended to be indicated.

According to the invention, the task is solved by the subject matters according to claims 1 and 7. Further advantageous configurations will result from the subclaims.

The task is in particular solved by a method for processing material, in particular for modifying material and/or material properties, by means of laser radiation, comprising the following steps:
a) generating a multiplicity of laser pulses,
b) controlling the point of impact of the laser pulses on a workpiece to be processed, in particular deflecting the laser pulses and/or moving the workpiece to be processed, such that the laser pulses are guided along a predetermined trajectory on the workpiece to be processed, wherein
   a pulse-to-pulse time interval between the individual laser pulses generated, and/or
   a pulse energy of the laser pulses, and/or
   a beam diameter of the laser pulses, and/or
   the predetermined trajectory
      is/are specifically subjected to noise.

The point of impact preferably is in each case a point of impact of an individual laser pulse. Consequently, in step b), a plurality of points of impact of a plurality, in particular subsequent laser pulses are controlled. In other words, in each case one point of impact of an individual laser pulse may be controlled in step b), wherein a plurality of points of impact of a plurality of laser pulses is controlled in a temporally consecutive manner. The points of impact may be processing points generated by laser pulses, for example.

An essential core idea of the invention is to influence the processing and/or modifying of the material of the workpiece such that, following the processing, a processed surface of the workpiece generates only small, preferably no optical effects such as diffraction effects, for example, under light incidence of coherent or incoherent light.

This is achieved in that an irregular structure of the modification of the material of the workpiece resulting from individual laser pulses specifically subjected to noise or by varying several parameters of the laser unit does not lead to any interference phenomena or diffraction phenomena under the incidence of light onto the workpiece, or rather the interference phenomena or diffraction phenomena will average out due to the generated irregular surface structure.

By specifically subjecting individual or a plurality of parameters, such as the trajectory of the laser beam, the pulse-to-pulse time interval, or the pulse energy, or the beam diameter to noise, achieves that the processing laser beam generates a deliberate irregular structure on the workpiece modified by the laser pulses.

Light impinging on the generated irregular structure of the workpiece after the processing operation, is scattered diffusely, and, on the workpiece, there are no diffraction phenomena or interference phenomena in reflection or transmission such as in an optical grating or other objects having a very regular, periodic structure or periodically structured surface, for example.

The method according to which individual parameters of the laser pulses are specifically subjected to noise so as to generate an irregular structure on the workpiece, is realizable in a cost-efficient manner according to one embodiment of the invention, since a workpiece does not need to be moved mechanically according to this embodiment. In this case, the resulting irregular structure on the surface of the workpiece may be adjusted to be arbitrarily small, with the processing of material not being limited by limited mechanical precession, such as, for example, hysteresis effects. On the other hand, this embodiment is suited for workpieces that cannot be moved, since they are in a rigid connection with heavy or immovable bodies, for example. The method for processing material is in this case processed such that no diffraction phenomena or interference phenomena will occur on the workpiece after processing material, when light impinges on the workpiece.

In the meaning of the invention, noise may be understood to be a stochastic signal or a time-discrete stochastic signal sequence, which is based on a stochastic noise process, such as, for example, a Gauss process, a Poisson process, white noise or an evenly distributed random process.

Specifically subjecting to noise may be understood in this context such that characteristic parameters of a fundamental noise process are specifically selected, such as, for example, an average value and/or a variance in a Gauss Process, and a stochastic signal or a time-discrete stochastic signal sequence is generated based on the noise process.

The generated stochastic signal or the generated time-discrete stochastic signal sequence can be appended, for example, additively to the control signal for controlling the point of impact of the laser pulses.

The noise may be understood in particular to be jitter, in particular in the meaning of temporal noise, which may likewise be based on a noise process, for example.

In a further and/or alternative embodiment of the invention, controlling the point of impact of the laser pulses on the workpiece is performed by moving the workpiece to be processed. In doing so, it is possible to use a workpiece holder, in particular a coordinate table, which is movable in three directions (x direction, y direction, z direction). By means of moving the workpiece to be processed in such a manner, it is possible to do without a more complex laser deflection system.

A temporal pulse duration may in his case—depending on the application—preferably be in the range of nanoseconds (ns), further preferred in the range of picoseconds (ps), still further preferred in the range of femtoseconds (fs), with the temporal course of the laser pulses being preferably distinct in the Gauss or sech² shape.

A wavelength of the laser pulses used in the method may be in this case—depending on the application, material and/or the desired penetration depth into the workpiece—in the ultraviolet range (UV), preferably in the visible range (VIS), still further preferred in the near infrared range (NIR).

A spatial mode of the laser pulses used in the method is preferably a TEM₀₀ mode, wherein this mode may also deviate from this mode in alternative embodiments.

An average pulse-to-pulse time interval Δt of the laser pulses L may preferably be in a range between 1 kHz and 100 MHz.

An average pulse energy Pi of the laser pulses L may preferably be in a range of 0.1 nJ to 1 mJ.

In one embodiment of the method, the pulse-to-pulse time interval is varied by a pseudo random pulse interval sequence of a determined pulse sequence length, wherein the pseudo random pulse interval sequence in particular is cyclically repeated. The pseudo random pulse interval sequence allows the pulse sequence to be effectively subjected to a jitter, namely temporal noise.

By generating the pseudo random pulse interval sequence, it is achieved that a continuous deflecting of the laser pulses causes irregular impact coordinates or points of impacts of the trajectory of the laser pulses on the workpiece. This results in an irregular modification of the material of the workpiece in material processing.

In a preferred embodiment of the method, a pseudo random pulse interval sequence is defined, which is cyclically repeated, wherein a start pulse of the repeating pseudo random pulse interval sequence is temporally shifted by a pseudo random value.

Cyclically repeating a pseudo random pulse interval sequence reduces the computational effort, since new pseudo random pulse interval sequences do not need to be computed continuously.

In one embodiment of the method, the pulse energy of the laser pulses is varied by a pseudo random pulse interval sequence, wherein in particular the pseudo random pulse interval sequence is cyclically repeated.

Varying the pulse energy of the laser pulses allow similar effects to be achieved as in the preceding embodiments. It may be advantageous, for example, depending on the material or optical properties of the workpiece, not to vary the pulse interval but rather the pulse energy. This is advantageous in case of materials, for example, which strongly reflect in laser wavelength.

In a further embodiment of the method, the pulse diameter of the laser pulses L is varied by means of a pseudo random pulse diameter sequence, which is in particular cyclically repeated.

Here, as well, the cyclical repetition of merely a pseudo random pulse diameter sequence reduces the computational effort since new pseudo random pulse diameter sequences do not need to be computed.

In one embodiment of the method, points of the predetermined trajectory along which the laser pulses are guided, are shifted by a pseudo random pulse trajectory sequence, wherein in particular the pseudo random pulse trajectory sequence is cyclically repeated.

The pseudo random shifting of the trajectory of the laser pulses allows a regular structure to be likewise avoided. This embodiment represents a simply employable mechanical embodiment, which is in particular combinable with the embodiments above.

It is possible for the laser unit to include the seed laser. In such an embodiment of the invention it is possible for such a noise, in particular such a jitter, to be selected, which is smaller than the period duration of the seed laser.

The task according to the invention is solved in a further aspect of the invention by a device for processing material, in particular for modifying material and/or material properties, by means of laser radiation, preferably for executing the method described above, wherein the device includes the following:
a laser unit for generating a multiplicity of laser pulses;
a unit for controlling the point of impact of the laser pulses on a workpiece to be processed, in particular a deflecting unit for deflecting the laser pulses along a predetermined trajectory and/or a movement unit for moving the workpiece to be processed, such that the laser pulses are guided along a predetermined trajectory on the workpiece to be processed;
optionally a displaying unit for displaying, in particular for focusing, the laser pulses along the predetermined trajectory on a workpiece to be processed; as well as
a system controller in communicable connection with the laser unit and/or the unit for controlling the point of impact of the laser pulses, in particular the deflecting unit and/or the movement unit and/or the displaying unit, such that the laser unit and/or the unit for controlling the point of impact of the laser pulses (L), in particular the deflecting unit (20) and/or the movement unit and/or the displaying unit, is/are controllable by the system controller by means of control signals, wherein:
a pulse-to-pulse time interval between the individual laser pulses generated, and/or
a pulse energy of the laser pulses, and/or
a beam diameter of the laser pulses, and/or
the predetermined trajectory
   is/are variable by specifically subjecting at least one of the control signals to noise.

The deflecting unit may be based on a galvanometer scanner, for example. In this case, deflecting of the laser pulses is accompanied by varying the impact coordinates or points of impact of the laser pulses on the workpiece.

For example, a telescope or a lens or a lens array or a lens arrangement or a parabolic mirror or a spherical mirror may be understood to be a displaying unit.

A focusing unit, for example, may be understood to be a displaying unit. Further, simple lens arrangements are also possible as a displaying unit.

A laser unit, for example, may be composed at least of a seed laser, a reinforcing fiber, an acousto-optical modulator (AOM) and an electro-optical modulator (EOM). The system controller, for example, requests a start pulse. A pulse emission is performed after the reinforcement by switching the EOM and the AOM into a conducting, in particular open state. In detail, reinforcement builds up when the EOM is switched into a non-conducting, in particular closed state.

As soon as a desired reinforcement is reached, the pulse emission is performed in that the EOM and the AOM are switched into a conducting, in particular open state. When no pulse is being requested, the EOM is in an open state and the AOM is in a closed state. In other words, the AOM blocks a pulse emission of the seed laser, and since the EOM is in an open state, no reinforcement will build up.

The control signals may be transistor-transistor logic (TTL) signals, for example.

It should be pointed out that the embodiments described above are arbitrarily combinable.

Hereinafter, the invention will be described also with respect to further features and advantages on the basis of exemplary embodiments, which will be explained in more detail by means of the Figures. Shown are in:

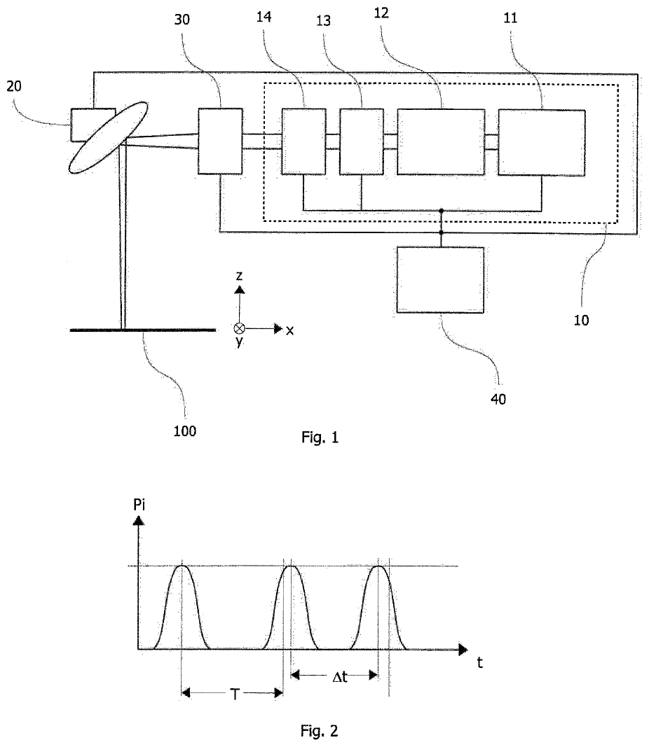

FIG. 1: a schematic representation of the device according to an exemplary embodiment of the invention for processing material of a workpiece;

FIG. 2: a schematic representation of the pulse sequence according to an exemplary embodiment of the invention;

FIG. 3: a top view of a workpiece during processing according to the state of the art;

FIG. 4: a top view of a workpiece during a processing method according to an exemplary embodiment according to the invention; as well as

FIG. 5: a schematic flow chart according to an exemplary embodiment of the invention.

In FIG. 1, a laser unit 10 is shown, which includes a seed laser unit 11 designed to emit light pulses in the direction of an amplifier area 12, wherein a first optical modulator 13, in particular an electro-optical modulator (EOM), is inferior to the amplifier area 12, which modulator has a first state causing light pulses to be able to leave the amplifier area, and a second state in which the light pulses circulate within the amplifier area so as to be amplified there per circulation.

Furthermore, the laser unit 10 comprises, downstream of the first optical modulator 13, a second optical modulator 14, in particular an acousto-optical modulator (AOM), which has a first state causing light pulses to be emitted from the laser unit 10, and a second state causing light pulses of the laser unit 10 not to be emitted and remaining in it.

Light pulses emitted from the laser unit 10 are displayed by a displaying unit 30, in particular of a focusing unit arranged downstream of the laser unit 10, along a predetermined trajectory Z in the direction of a workpiece 100 to be processed. The displaying unit 30 may in this case be a telescope, a lens, a lens array, a parabolic mirror or a spherical mirror, or a combination of two or more of these elements.

For influencing the beam position or the point of impact on the workpiece 100, a deflecting unit 20 is located between the displaying unit 30 and the workpiece. The deflecting unit 20 serves the purpose of deflecting the laser pulses L along a predetermined trajectory Z on the workpiece 100 to be processed. In FIG. 1, the average angle of deviation of the beam is approximately 90°. According to the invention, the device, however, is not restricted to this average angle of deviation.

Furthermore, a coordinate system x, y and z is illustrated in FIG. 1, wherein the x-axis and y-axis span the plane of the workpiece 100, and the z-axis runs orthogonally to the workpiece plane.

A system controller 40 determines via the first modulator 13 and the second modulator 14, at which points in time a laser pulse is emitted from the laser unit, controls the displaying unit 30 with respect to a focus position relative to the workpiece 100, and controls the predetermined trajectory Z of the laser pulses via the deflecting unit 20, and thus controls the beam position with respect to the workpiece 100.

In doing so, the system controller is able to specifically subject parameters of the pulse interval between the individual generated laser pulses and/or a pulse energy of the laser pulses and/or a beam diameter of the laser pulses and/or the predetermined trajectory to noise so as to avoid the problems in processing material mentioned before.

In a further exemplary embodiment, it is also possible for the system controller to subject the temporal emission of the light pulses of the seed laser 11 to noise.

In FIG. 2, a schematic representation of a pulse sequence according to one exemplary embodiment of the invention is illustrated. The pulse energy Pi over time t is depicted. The individual pulses are at least substantially Gauss pulses having a constant energy level in the present example. Furthermore, a repetition rate T is delineated in FIG. 2, which represents the period duration in which the individual laser pulses are repeated. In the exemplary embodiment shown in FIG. 2, the pulse intervals are varied by means of a pseudo random pulse interval sequence such that the single pulse intervals Δt are varied from pulse to pulse.

In FIG. 4, a top view of a workpiece 100 during processing of the workpiece 100 according to the inventive method is represented. In the exemplary embodiment shown, a pulsed laser beam is guided along a trajectory Z on a surface of the workpiece 100.

In this exemplary embodiment, trajectory Z runs along an x-axis of a coordinate system (x, y, z), wherein the x-axis and the y-axis span a workpiece plane of the workpiece and the z-axis runs orthogonally to the x-axis and the y-axis. Depending on focussing and quality of the material of the workpiece, it is possible for the pulsed laser beam to be guided along a trajectory Z within the workpiece.

In FIG. 4, the processing points L generated by the laser pulses are moreover shown, which have a diameter D. Since the pulsed laser beam is guided and/or deflected continuously along trajectory Z, but the time intervals between the laser pulses vary temporally, an irregular pattern of processing points L results on the workpiece.

Likewise, further parameters of the laser pulses can be specifically varied, in particular additionally, for example by means of pseudo random pulse energy sequences, pulse diameter sequences and/or pulse trajectory sequences.

FIG. 5 shows a flowchart according to one exemplary embodiment of the invention. In step S0, a control signal is generated, which is specifically subjected to temporal noise, in particular so-called jitter. The control signal may be a TTL signal, for example, which has low voltage values, for example below 0.2 V, and high voltage values, for example above 2 V.

The control signal specifically subjected to jitter is transmitted to the laser unit 10 in step S1. In step S2, it is decided in the laser unit 10 by means of the control signal whether a laser pulse should be applied. This may be triggered by a threshold value comparison, for example when the voltage value of the control signal is above 1.8 V.

When in step S2 a decision has been made that a laser pulse should be triggered, the EOM of the laser unit 10 is closed in step S3. Thereby, an inversion, i.e. a reinforcement of the laser signal of the seed laser unit 11 builds up in the laser unit 10. If, on the contrary, in step S2, for example when the voltage value of the control signal is below 1.8 V, a decision is made that no laser pulse should be triggered, the method returns to step S1.

In step S4, a query is made as to whether a certain inversion time has passed, so that a desired reinforcement of the laser signal of the seed leaser unit 11 has been reached.

If the certain inversion time has passed, both the EOM and the AOM are opened in step S5. Hereby, a laser pulse is now emitted from the laser unit 10 and applied to the material to be processed.

In a further step S6, the EOM is closed after the laser pulse has been emitted. After closing the EOM, the AOM will also be closed, with the closing process of the AOM, however, being slower than the closing process of the EOM.

The method returns now to step S2, in which a decision is made by means of the trigger signal, if a laser pulse should be emitted again.

Independent of the exemplary embodiments represented in FIGS. 1 to 5, reference is made to still a further possible field of application of the invention: A possible field of application of the method according to the invention and/or the device according to the invention, for example, is the treatment of the cornea of an eye, in particular a human eye, by a laser, in particular an ultrashort pulse (USP) laser.

During the methods known from the state of the art for treating the cornea of an eye, a periodic structure is hereby formed as a result. These methods are inter alia employed in the application Femto-LASIK (Laser-assisted in situ keratomileusis supported by femtosecond lasers) and the application FLEX (femtosecond laser lenticular extraction).

The laser, in particular the ultrashort pulse laser thereby generates a structure of cavitation bubbles in the tissue so that tissue parts can be subsequently separated from one another along the generated separation layers.

The diffraction effects resulting from the periodic structure of the treatment, subsequent to the treatments according to the state of the art, result for the patient in the perception of a rainbow structure when looking at bright light sources. The effect is known under the designation “rainbow glare” as a side effect of the above-mentioned methods of the state of the art.

By means of the present invention, a method and/or a device are/is provided by means of which the separation layers can be generated without forming a periodic structure in the tissue in the process. This results in suppressing the diffraction effects and thus in reducing the side effect of the above-mentioned applications.

In other words, a method for processing a cornea of an eye, in particular a human eye, by means of laser radiation, comprising the steps mentioned in method claim 1, is proposed in a possible embodiment of the invention. The eye is in this case defined to be the workpiece to be processed.

In further embodiments of the invention, the method steps mentioned in the subclaims are also applied in processing the cornea of the eye.

A further aspect of the invention relates to a device for processing a cornea of an eye, in particular a human eye, by means of laser radiation, comprising the components mentioned in claim 7. The eye is in this case defined to be the workpiece to be processed.

### LIST OF REFERENCE NUMERALS

10 laser unit
11 seed laser unit
12 amplifier area
13 first optical modulator
14 second optical modulator
20 deflecting unit
30 displaying unit (e.g. focussing unit)
40 system controller
100 workpiece
D beam diameter
D′ beam diameter of the laser pulses
L laser pulses (processing points generated by the laser pulses)
L′ laser pulses (processing points generated by the laser pulses) of the state of
the art
Pi pulse energy
S0 generating a control signal subjected to noise
S1 transmitting the control signal to the laser unit
S2 querying whether a laser pulse should be applied
S3 building up the reinforcement of the seed laser unit (inversion)
S4 querying whether a desired inversion time has been reached
S5 opening the EOM and the AOM
S6 closing the EOM and (including a delay) the AOM
Δt pulse interval
T repetition rate
Z trajectory
Z′ trajectory of the state of the art

1. A method for processing material, in particular for modifying material and/or material properties, by means of laser radiation, comprising the following steps:
a) generating a multiplicity of laser pulses (L);
b) controlling the point of impact of the laser pulses (L) on a workpiece (100) to be processed, in particular deflecting the laser pulses (L) and/or moving the workpiece (100) to be processed, such that the laser pulses (100) are guided along a predetermined trajectory (Z) on the workpiece (100) to be processed;
characterized in that
a pulse-to-pulse time interval (Δt) between the individual laser pulses (L) generated, and/or
a pulse energy (Pi) of the laser pulses (L), and/or
a beam diameter (D) of the laser pulses (L), and/or
the predetermined trajectory (Z)
is/are specifically subjected to noise.

2. The method according to claim 1,
characterized in that
the pulse-to-pulse time interval (Δt) is varied by a pseudo random pulse interval sequence (Ppt) of a determined pulse sequence length (LI), wherein in particular the pseudo random pulse interval sequence (Ppt) is cyclically repeated.

3. The method according to claim 2,
characterized in that merely a pseudo random pulse interval sequence (Ppt) is defined, which is cyclically repeated, wherein a start pulse of the repeating pseudo random pulse interval sequence (Ppt) is temporally shifted by a pseudo random value.

4. The method according to claim 1,
characterized in that
the pulse energy (Pi) of the laser pulses (L) is varied by a pseudo random pulse energy sequence (Ppe), wherein in particular the pseudo random pulse energy sequence (Ppe) is cyclically repeated.

5. The method according to claim 1,
characterized in that
the beam diameter (D) of the laser pulses (L) is varied by means of a pseudo random pulse diameter sequence (Ppd), which is in particular cyclically repeated.

6. The method according to claim 1,
characterized in that
points of the predetermined trajectory (Z), along which the laser pulses (L) are guided, are shifted by a pseudo random pulse trajectory sequence (Ppz), wherein in particular the pseudo random pulse trajectory sequence (Ppz) is cyclically repeated.

7. A device for processing material, in particular for modifying material and/or material properties, by means of laser radiation, preferably for executing the method according to claim 1,
wherein the device includes the following:
a laser unit (10) for generating a multiplicity of laser pulses (L);
a unit for controlling the point of impact of the laser pulses (L) on a workpiece (100) to be processed, in particular a deflecting unit (20) for deflecting the laser pulses (L) along a predetermined trajectory (Z) on the workpiece (100) to be processed and/or a movement unit for moving the workpiece to be processed;
optionally a displaying (30) unit for displaying, in particular for focusing, the laser pulses (L) along the predetermined trajectory (Z) on a workpiece (100) to be processed; as well as
a system controller (40) in communicable connection with the laser unit (10) and/or the unit for controlling the point of impact of the laser pulses (L), in particular the deflecting unit (20) and/or the movement unit and/or the displaying unit (30), such that the laser unit (10) and/or the unit for controlling the point of impact of the laser pulses (L), in particular the deflecting unit (20) and/or the movement unit and/or the displaying unit (30), is/are controllable by the system controller (40) by means of control signals, wherein:
a pulse-to-pulse time interval (Δt) between the individual laser pulses (L) generated, and/or
a pulse energy of the laser pulses (L), and/or
a beam diameter (D) of the laser pulses (L), and/or
the predetermined trajectory (Z)
is/are variable by specifically subjecting at least one of the control signals to noise.
